(19) United States
(12) Patent Application Publication
(10) Pub. No.: US 2020/0063107 A1
(43) Pub. Date: Feb. 27, 2020

(54) BLOOD VESSEL MIMIC AND METHOD FOR CULTURING BLOOD VESSEL MIMIC

(71) Applicant: POSTECH ACADEMY-INDUSTRY FOUNDATION, Pohang-si (KR)

(72) Inventors: Jeong Sik KONG, Bucheon-si (KR); Dong Woo CHO, Seoul (KR); Ge GAO, Pohang-si (KR)

(21) Appl. No.: 16/610,625

(22) PCT Filed: May 10, 2018
(86) PCT No.: PCT/KR2018/005336

§ 371 Date: Nov. 4, 2019

(30) Foreign Application Priority Data
May 12, 2017 (KR) 10-2017-0059309

(51) Int. Cl. C12N 5/071; C12M 3/00; B33Y 10/00; B33Y 70/00; B33Y 80/00

(52) U.S. Cl. CPC C12N 5/0691; C12M 21/08; B33Y 10/00; B33Y 70/00; C12N 2533/00; C12N 2513/00; C12N 2537/10; C12N 2533/18; C12N 2533/92; B33Y 80/00

(57) ABSTRACT

A method for culturing a blood vessel mimic according to an embodiment of the present invention comprises the steps of: printing a lower structure of a chamber; printing a blood vessel mimic on the lower structure; printing an upper structure of the chamber on the lower structure and the blood vessel mimic; connecting, to both ends of the blood vessel mimic, tubes connected to a circulating pump, respectively; and operating the circulating pump to circulate a fluid through the blood vessel mimic.

### TECHNICAL FIELD

The research related to the present invention was carried out by the support of the ICT Convergence Original Technology Development Project (Project Title: Development and Commercialization of Artificial Skin Model Using 3D Bioprinting for Substitution of Animal Experiment, Project No.:1711061192) under the supervision of the Ministry of Science and ICT.

The present invention relates to a blood vessel mimic and a method for culturing a blood vessel mimic, and more specifically, to a blood vessel mimic and a method for culturing a blood vessel mimic using 3D printing.

### BACKGROUND ART

For the treatment of cardiovascular disease, research has been conducted on the preparation of a vascular replacement that can be used for bypass surgery.

Recently, artificial blood vessels made of materials, such as polyethylene terephthalate (Dacron) and polytethrafluoroethylene (Teflon), have been used, but these artificial blood vessels have a disadvantage in that the blood flow rate decreases as the diameter of the blood vessel decreases.

To prepare blood vessel replacements with a size less than 6 mm in diameter, recently, methods for preparing a blood vessel mimic via tissue engineering using a cell plate technology, an organ decellularization technology, etc. are being studied.

### DISCLOSURE

### Technical Problem

The objects to be achieved in the present invention is to provide a blood vessel mimic that closely resembles a real blood vessel and a method for culturing the blood vessel mimic.

The objects of the present invention are not limited to the above-mentioned objects, and other objects not mentioned will be clearly understood by those skilled in the art from the description herein below.

### Technical Solution

To achieve the above objects, an embodiment according to the present invention provides a method for culturing a blood vessel mimic, which includes the steps of: printing a lower structure of a chamber; printing a blood vessel mimic on the lower structure; printing an upper structure of the chamber on the lower structure and on the blood vessel mimic; connecting, to both ends of the blood vessel mimic, tubes connected to a circulating pump, respectively; and operating the circulating pump to circulate a fluid through the blood vessel mimic.

The lower structure may include a seating part on which the blood vessel mimic is seated.

In the step of printing a blood vessel mimic, the blood vessel mimic may be printed such that both ends of the blood vessel mimic protrude from the seating part to the outside of the seating part.

The upper structure may include a fixing part which is extended from the seating part such that both ends of the blood vessel mimic are fixed to the seating part.

In the step of printing an upper structure of the chamber, the fixing part may be printed such that both ends of the blood vessel mimic protrude to the outside of the fixing part.

The lower structure may further include a lower frame that encompasses both ends of the blood vessel mimic along with the seating part, and the upper structure may further include an upper frame which is extended from the lower frame and encompasses both ends of the blood vessel mimic along with the fixing part.

The method may further include a step of filling a filling material for fixing the blood vessel mimic into a space, which is encompassed with the lower frame, the upper frame, the seating part, and the fixing part.

The filling material may be silicone oil.

The method, after the filling material is filled, may further include a step of hardening of the filling material.

The method may further include a step of forming, on the hardened filling material, a hole to be connected to both ends of the blood vessel mimic, and in the step of connecting the tubes, the tubes may be inserted into the hole and connected to both ends of the blood vessel mimic.

The blood vessel mimic, which is printed in the step of printing the blood vessel mimic, may include: a solution in which calcium ions are dissolved; a first layer, which encompasses the solution along the longitudinal direction of the blood vessel mimic and is crosslinked while reacting with the calcium ions; and a second layer, which encompasses the first layer along the longitudinal direction of the blood vessel mimic and is crosslinked while reacting with the calcium ions.

The first layer may include a first bioink in which vascular endothelial cells and alginate are mixed with a decellularized extracellular matrix isolated from a blood vessel tissue; and the second layer may include a first bioink in which smooth muscle cells and alginate are mixed with a decellularized extracellular matrix isolated from a blood vessel tissue.

The method may further include a step of controlling the perfusion pressure of the fluid by controlling the circulating pump.

The first layer may be cultured with vascular endothelial cells, the second layer may be cultured with smooth muscle cells, the vascular endothelial cells may be arranged such that the flow direction of the fluid becomes the long axis, and the smooth muscle cells may be arranged such that the direction perpendicular to the flow direction of the fluid becomes the long axis.

In the step of circulating the fluid, the fluid may be introduced into the inside of the blood vessel mimic by the circulating pump and discharged from the blood vessel mimic along with the solution.

To achieve the above objects, an embodiment according to the present invention provides a blood vessel mimic, which includes: a first layer, which is printed so as to have a tubular shape using a first bioink in which vascular endothelial cells are mixed with a decellularized extracellular matrix isolated from a blood vessel tissue; and a second layer, which is printed so as to encompass a side of the first layer and have a tubular shape using a second bioink in which smooth muscle cells are mixed with a decellularized extracellular matrix isolated from a blood vessel tissue.

The blood vessel mimic may further include a core layer that is formed inside of the first layer and is printed using a solution in which calcium ions are dissolved.

The first bioink and the second bioink may further include alginate; and the calcium ions may react with the alginate as the core layer, the first layer, and the second layer are printed and thereby the first layer and the second layer may be crosslinked.

After the first layer and the second layer are crosslinked, the core layer may be removed by the fluid that flows through the first layer.

The core layer, the first layer, and the second layer may be printed through multiple coaxial nozzles; and the multiple coaxial nozzles may include: a first nozzle, in which the solution where the calcium ions are dissolved is extruded; a second nozzle, which is arranged concentrically to encompass the first nozzle and in which the first bioink is extruded; and a third nozzle, which is arranged concentrically to encompass the second nozzle and in which the second bioink is extruded. Other specific details of the invention are included in the Detailed Description and Drawings.

### Advantageous Effects

According to the embodiments, the present invention has at least the following effects.

It is possible to prepare a blood vessel mimic that closely resembles a real vessel.

The effects according to the present invention are not limited by the contents illustrated above, and more various effects are included in the present specification.

### DESCRIPTION OF DRAWINGS

FIG. 1 is a flow chart illustrating a method for culturing a blood vessel mimic according to an embodiment of the present invention.

FIG. 2 is a diagram for illustrating Step S11 of FIG. 1.

FIG. 3 is a diagram for illustrating Step S12 of FIG. 1.

FIG. 4 is a diagram for illustrating multiple coaxial nozzles used in Step S12.

FIG. 5 is a schematic diagram for illustrating the multiple coaxial nozzles of FIG. 4.

FIG. 6 is a schematic diagram for illustrating a blood vessel mimic which is printed by multiple coaxial nozzles.

FIG. 7 is a diagram for illustrating Step S13 of FIG. 1.

FIG. 8 is a diagram for illustrating Step S14 of FIG. 1.

FIG. 9 is a diagram for illustrating Step S16 of FIG. 1.

FIG. 10 is a diagram for illustrating Step S17 of FIG. 1.

### MODE FOR INVENTION

Advantages and features of the present invention, and methods for accomplishing the same will become apparent when referred to the embodiments described below in detail in conjunction with the accompanying drawings. However, the present invention is not limited to the embodiments disclosed below, but may be implemented in various different forms, and the embodiments are provided only to make the disclosure of the present invention complete, and to fully deliver the scope of the invention to those skilled in the art, and the invention is only defined by the scope of the claims. Like reference numerals refer to like elements throughout the specification.

In addition, the embodiments described herein will be described with reference to cross-sectional and/or schematic views, which are ideal illustrations of the invention. Accordingly, shapes of the exemplary views may be modified by manufacturing techniques and/or tolerances. In addition, each element in each drawing shown in the present invention may be shown to be somewhat enlarged or reduced in view of the convenience of description. Like reference numerals refer to like elements throughout the specification.

Hereinafter, the present invention will be described with reference to the drawings for illustrating a blood vessel mimic and a method of culturing a blood vessel mimic according to an embodiment of the present invention.

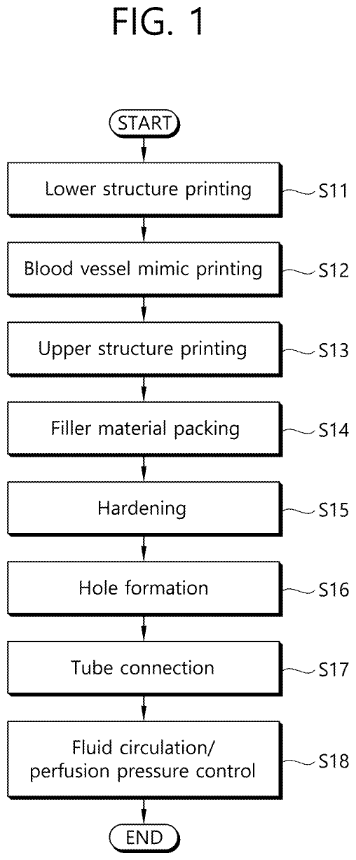

FIG. 1 is a flow chart illustrating a method for culturing a blood vessel mimic according to an embodiment of the present invention.

As illustrated in FIG. 1, the method for culturing a blood vessel mimic according to an embodiment of the present invention includes:

a step of printing a lower structure (S11), a step of printing a blood vessel mimic (S12), a step of printing an upper structure (S13), a step of filling a filling material (S14), a step of hardening the filling material, etc. (S15), a step of forming holes on the hardened filling material (S16), a step of connecting tubes to a blood vessel mimic through the holes (S17), and a step of circulating a fluid through the tubes and controlling a perfusion pressure of the fluid (S18).

The method for culturing a blood vessel mimic according to an embodiment of the present invention is performed using a three-dimensional (3D) printing system. The 3D printing system includes a 3D printer equipped with a plurality of printing heads controlled in the XYZ direction, and each of the printing heads may eject a synthetic polymer, a naturally occurring polymer, etc. by means of extrusion.

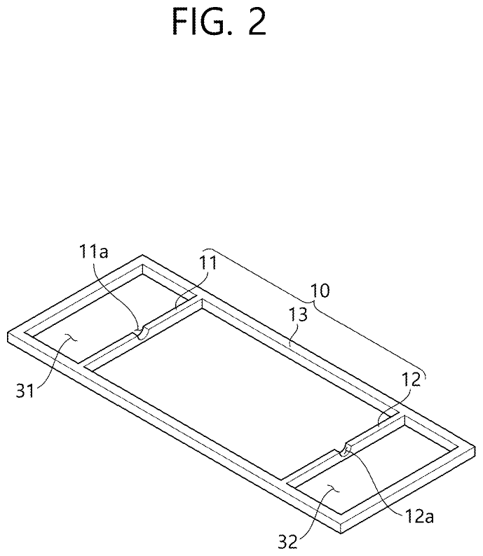

Hereinafter, each step will be described in detail with reference to the drawings of FIGS. 2 to 10.

FIG. 2 is a diagram for illustrating Step S11 of FIG. 1.

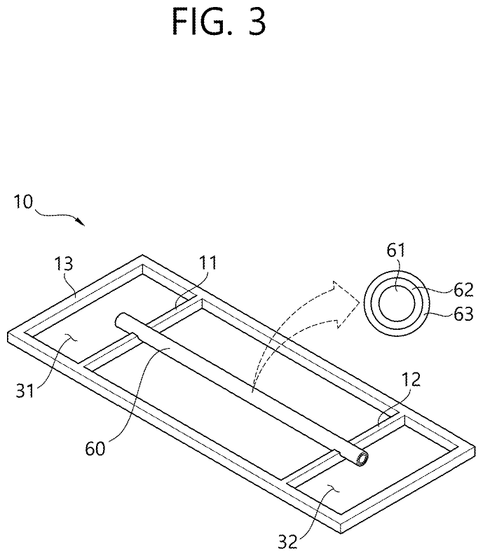

In the step of printing a lower structure (S11), a lower structure 10 of a chamber that fixes a blood vessel mimic 60 (see FIG. 3) is formed.

As illustrated in FIG. 2, the lower structure 10 includes a lower frame 13 having a substantially rectangular frame shape, and a first a seating part 11 and a second a seating part 12 that run side by side across the lower frame 13.

The first seating part 11 partitions one side within the lower frame 13 to form a first filling space 31, and the second seating part 12 partitions the other side within the lower frame 13 to form a second filling space 32.

In the center of the first seating part 11, a first seating groove 11a may be formed in which one side of a blood vessel mimic 60 (see FIG. 3) is seated and fixed, whereas in the center of the second seating part 12, a second seating groove 12a may be formed in which the other side of a blood vessel mimic 60 (see FIG. 3) is seated and fixed.

In Step S11, the 3D printing system moves the printing heads filled with a synthetic polymer, extrudes the synthetic polymer, and prints while stacking the first seating part 11, the first seating part 12, and the lower frame 13. As the synthetic polymer, polycarprolactone (PCL) may be used.

In this embodiment, an example in which the lower frame 13, the first filling space 31, and the second filling space 32 are formed in a substantially rectangular shape is illustrated, but the shape may vary depending on the embodiment.

FIG. 3 is a diagram for illustrating Step S12 of FIG. 1.

In the step of printing a blood vessel mimic (S12), a blood vessel mimic 60 is printed on the lower structure 10.

As illustrated in FIG. 3, the blood vessel mimic 60 is printed such that one side is located in a first seating groove 11a of the first seating part 11 and the other side is located in a second seating groove 12a of the first seating part 12. One end of the blood vessel mimic 60 protrudes to the outside of the first seating part 11 and is located within the first filling space 31, and the other end of a blood vessel mimic 60 protrudes to the outside of the second seating part 12 and is located within the second filling space 32.

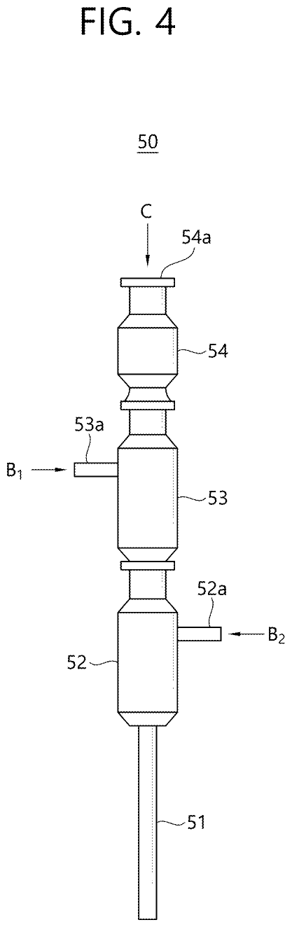

As illustrated in FIG. 3, the blood vessel mimic 60 is formed to have three layers 61, 62, and 63, for which the blood vessel mimic 60 is printed by multiple coaxial nozzles 50 (see FIG. 4).

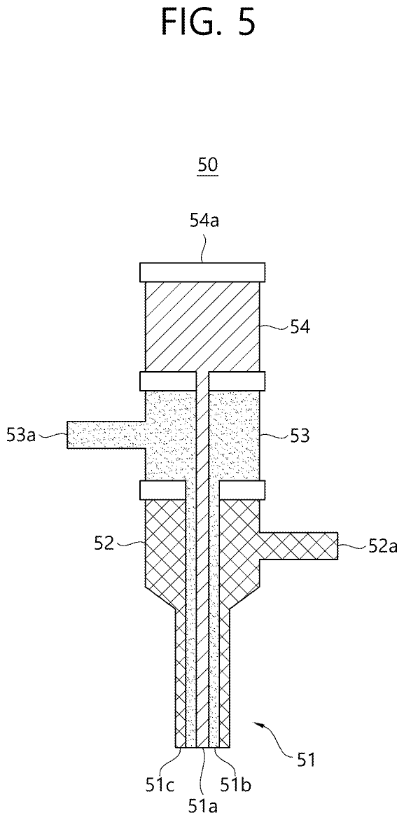

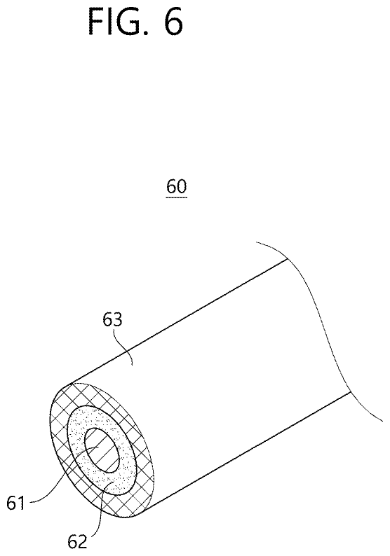

FIG. 4 is a diagram for illustrating multiple coaxial nozzles used in Step S12, FIG. 5 is a schematic diagram for illustrating the multiple coaxial nozzles of FIG. 4, and FIG. 6 is a schematic diagram for illustrating a blood vessel mimic which is printed by multiple coaxial nozzles.

As illustrated in FIG. 4, in the multiple coaxial nozzles 50, a nozzle part 51 is formed at the bottom thereof, a first receiving part 52 is provided on the top of the nozzle part 51, a second receiving part 53 is provided on the top of the first receiving part 52, and a third receiving part 54 is provided on the top of the second receiving part 53.

The first receiving part 52 includes a first inlet 52a that opens to a side, the second receiving part 53 includes a second inlet 53a that opens to a side, and the third receiving part 54 includes a third inlet 54a that opens to the top.

As illustrated in FIG. 5, the nozzle part 51 includes three nozzles 51a, 51b, and 51c disposed concentrically. The three nozzles 51a, 51 b, and 51c are called from the center a first nozzle 51a, a second nozzle 51b, and a third nozzle 51c from the center.

The first nozzle 51a is in fluid communication with the third inlet 54a and the third receiving part 54. Accordingly, the materials introduced through the third inlet 54a are extruded through the first nozzle 51a.

The second nozzle 51b is in fluid communication with the second inlet 53a and the second receiving part 53. Accordingly, the materials introduced through the second inlet 53a are extruded through the second nozzle 51b.

The third nozzle 51c is in fluid communication with the third inlet 54a and the third receiving part 54. Accordingly, the materials introduced through the third inlet 54a are extruded through the third nozzle 51c.

Accordingly, when materials which are different from each other are introduced through the first inlet 52a, a blood vessel mimic 60 is printed, in which the blood vessel mimic 60 consists of the second inlet 53a, and the third inlet 54a and extruded through the nozzle part 51, a core layer 61 which is formed of the material discharged from the first nozzle 51a, a first layer 62 which is formed with the material discharged from a second nozzle 51b so as to have a tubular shape encompassing the core layer 61, and a second layer 63 which is formed to have a tubular shape so as to encompass the first layer 62 with a material discharged from the third nozzle 51c.

In the method for culturing a blood vessel mimic according to an embodiment of the present invention in which the blood vessel mimic 60 is cultured into a blood vessel tissue, a solution (C) in which calcium ions are dissolved is used as a material introduced into the third inlet 54a, and bioinks B1 and B2, which are different from each other, are used as materials introduced into the first inlet 52a and the second inlet 53a.

As an example of a solution (C) in which calcium ions forming the core layer 61 are dissolved, CPF127 containing 40% Pluronic F127 in a calcium chloride solution may be used.

As a first bioink B1 that forms the first layer 62, one in which vascular endothelial cells and alginate are mixed with a decellularized extracellular matrix may be used, and as a second bioink B2 that forms the second layer 63, one in which smooth muscle cells and alginate are mixed with a decellularized extracellular matrix may be used.

The decellularized extracellular matrix used to prepare the first bioink B1 and the second bioink B2 may be derived from a blood vessel tissue. In the present embodiment, a vascular decellularized extracellular matrix (VdECM) was prepared, in which extracellular matrix of vascular tissues (e.g., collagen, GAGs, and elastin) are preserved by physical, chemical, and enzymatic treatments of a porcine aorta while genes thereof are removed.

The process of preparing VdECM is as follows.

The tissue of a porcine aorta is sliced into a size of approximately 2 mm*2 mm*2 mm and washed with 0.3% sodium dodecyl sulfate (SDS), 3% Triton, 25 U/mL, DNase, etc. to remove the cells in the tissue.

Then, the resultant is dissolved in an acid solution where 0.5 M acetic acid and 0.6 wt % of pepsin are mixed and freeze-dried to obtain 60 mg/mL VdECM pre-gel.

Then, the VdECM pre-gel is neutralized with 10 M NaOH and thereby a vascular tissue-specific VdECM bioink is prepared.

Since the first bioink B1 and the second bioink B2 contain alginate and the solution C that forms the core layer 61 contains calcium ions, the alginate contained in the first layer 62 and the second layer 63, upon extrusion of the first layer 62 and the second layer 63 from the nozzle part 51, reacts with the calcium ions included in the core layer 61 and thereby a primary crosslinking is formed therebetween.

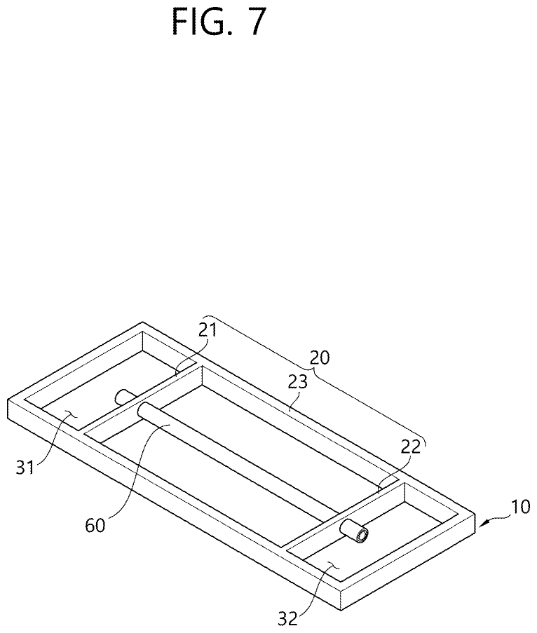

FIG. 7 is a diagram for illustrating Step S13 of FIG. 1.

In the step of printing an upper structure (S13), an upper structure 20 of a chamber is formed.

As illustrated in FIG. 7, the upper structure 20 is printed in such a way as to extend the lower structure 10 upwards.

More specifically, the upper structure 20 includes an upper frame 23 which is formed by extending upward from the lower frame 13, a first fixing part 21 which is formed by extending upward from the first seating part 11, and a second fixing part 22 which is formed by extending upward from the first seating part 12.

The first fixing part 21 and the second fixing part 22 are formed such that one end of the blood vessel mimic 60 protrudes to the outside of the first fixing part 21 and the other end protrudes to the outside of the second fixing part 22.

The first fixing part 21 is formed to cover one side of the blood vessel mimic 60, and the second fixing part 22 is formed to cover the other side of the blood vessel mimic 60. Accordingly, one side of the blood vessel mimic 60 is fixed between the first fixing part 21 and the first seating part 11, and the other side is fixed between the second fixing part 22 and the first seating part 12.

In Step S13, the 3D printing system moves the printing heads filled with a synthetic polymer, extrudes the synthetic polymer, and prints while stacking the first fixing part 21, the second fixing part 22, and the upper frame 23. Polycarprolactone (PCL) may be used as the synthetic polymer.

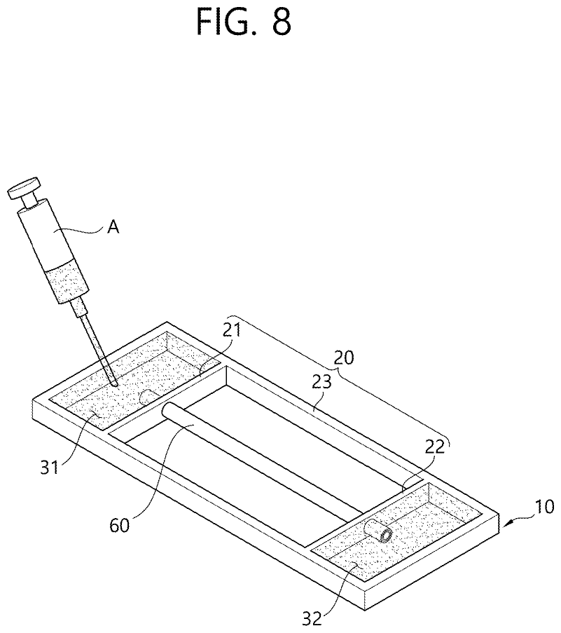

FIG. 8 is a diagram for illustrating Step S14 of FIG. 1.

As illustrated in FIG. 8, in the step of filling a filling material (S14), the filling material is filled into a first filling space 31 and a second filling space 32.

As the filling material, a material that can be hardened to be transparent enough to be observed at both ends of the blood vessel mimic 60 from the outside may be used. In this embodiment, PDMS (i.e., a silicone oil) was used.

The filling material may be filled into the first filling space 31 and the second filling space 32 using a separate injection tool (A) (e.g., syringes and pipettes).

In the step of hardening the filling material (S15), chambers 10 and 20, the blood vessel mimic 60, and a filling material are hardened. In this embodiment, the chambers were hardened at an atmosphere of about 37° C.

During the progress of Step S15, the filling material filled into the first filling space 31 and the second filling space 32 are hardened and fix both ends of the blood vessel mimic 60 within the first filling space 31 and the second filling space 32.

Then, the first layer 62 and the second layer 63 of the blood vessel mimic 60 are secondarily crosslinked.

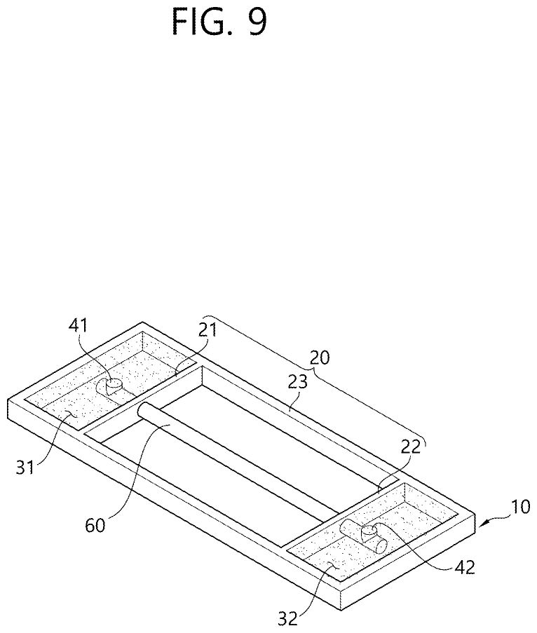

FIG. 9 is a diagram for illustrating Step S16 of FIG. 1.

As illustrated in FIG. 9, in the step of forming holes on the hardened filling material (S16), holes 41 and 42 are formed on the hardened filling material that is to be connected to both ends of the blood vessel mimic 60. Since both ends of the blood vessel mimic 60 are each located in the first filling space 31 and the second filling space 32, the holes 41 and 42 can be formed from the top of the first filling space 31 and the second filling space 32 towards both ends of the blood vessel mimic 60.

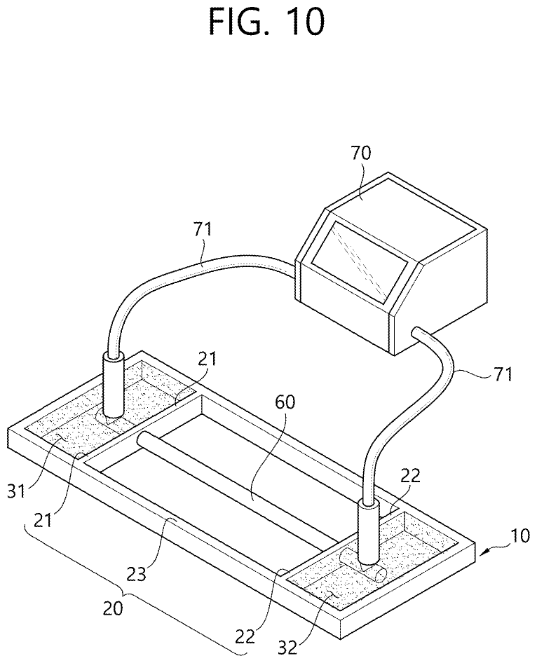

FIG. 10 is a diagram for illustrating Step S17 of FIG. 1.

As illustrated in FIG. 10, in the step of connecting tubes to the blood vessel mimic through the holes (S17), the tubes 51 which are connected to a pump 52 is connected to the holes 41 and 42. The tubes 51, the blood vessel mimic 60, and a pump 70 together form a closed loop.

In the step of circulating a fluid through tubes and controlling a perfusion pressure of a fluid (S18), the pump 70 is operated to supply to the blood vessel mimic 60 through tubes 71. That is, the pump 70 can supply a fluid to the blood vessel mimic 60 by flowing the fluid into tubes 71 connected to one end (or the other end) of the blood vessel mimic 60, and can circulate the fluid in such a way that the fluid which is discharged to the other end (or one end) of the blood vessel mimic 60 is recovered through the tubes 71 connected to the other end (or one end) of the blood vessel mimic 60.

The fluid supplied to the blood vessel mimic 60 via the tubes 71 dissolves a core layer 61, which is formed of a solution of calcium ions (C), and escapes from the blood vessel mimic 60. Then, when the fluid is flowed continuously, a first layer 62 is incubated with vascular endothelial cells and a second layer 63 is incubated with smooth muscle cells.

The circulating fluid can be selected as a culture medium suitable for the culture of vascular endothelial cells and smooth muscle cells. For example, as a culture medium, a mixture of C-22022 and C-22062 or a mixture of C-22022 and C-22062 may be used. The mixing ratio may be 1:1 when C-22022 and C-22062 are mixed.

Meanwhile, by controlling a perfusion pressure of the fluid using the pump 70, the vascular endothelial cells cultured in the first layer 62 can be cultured such that the flow direction of the fluid (i.e., the longitudinal direction of a blood vessel mimic 60) becomes the long axis, whereas the smooth muscle cells cultured in the second layer 63 can be cultured such that the direction perpendicular to the flow direction of the fluid becomes the long axis. This is the same as the arranged directions of vascular endothelial cells and smooth muscle cells in real blood vessels.

As described above, the blood vessel mimic according to an embodiment of the present invention is formed such that vascular endothelial cells form the lumen and smooth muscle cells encompass the vascular endothelial cells in an almost the same manner as in the actual vessels, and in addition, it is possible to simulate the directions of cell arrangement of vascular endothelial cells and smooth muscle cells to be almost the same as in the actual blood vessels.

Additionally, it is also possible to prepare a blood vessel mimic having a diameter of several millimeters to micrometers, depending on the shape of the nozzle of the multiple coaxial nozzles.

Additionally, the blood vessel mimic according to an embodiment of the present invention can be cultured stably in a fixed state by preparing a blood vessel mimic, and a chamber that can stably supply the culture solution to the blood vessel mimic through a 3D printing system.

Those skilled in the art will appreciate that the present invention can be embodied in other specific forms without altering the technical spirit or essential features of the present invention. Therefore, it should be understood that the embodiments described above are exemplary and not restrictive in all respects. The scope of the present invention is illustrated by the following claims rather than the detailed description, and all changes or modifications derived from the meaning and scope of the claims and their equivalents should be construed as being included in the scope of the present invention.

### MODE FOR CARRYING OUT THE INVENTION

A method for culturing a blood vessel mimic according to an embodiment of the present invention includes the steps of: printing a lower structure of a chamber; printing a blood vessel mimic on the lower structure; printing an upper structure of the chamber on the lower structure and the blood vessel mimic; connecting, to both ends of the blood vessel mimic, tubes connected to a circulating pump, respectively; and operating the circulating pump to circulate a fluid through the blood vessel mimic.

1. A method for culturing a blood vessel mimic, which comprises the steps of:
printing a blood vessel mimic, such that a solution in which calcium ions are dissolved forms a core layer; a tubular first layer that encompasses the core layer is formed using a first bioink in which vascular endothelial cells and alginate are mixed with a decellularized extracellular matrix isolated from a blood vessel tissue; and a tubular second layer that encompasses the first layer is formed using a second bioink, in which smooth muscle cells and alginate are mixed with a decellularized extracellular matrix isolated from a blood vessel tissue;
connecting, to both ends of the blood vessel mimic, tubes connected to a circulating pump, respectively; and
operating the circulating pump to circulate a fluid through the blood vessel mimic through the core layer.

2. The method of claim 1, wherein, in the printing a blood vessel mimic, the first layer and the second layer are crosslinked by reacting with the calcium ions.

3. The method of claim 1, wherein the method further comprises controlling the perfusion pressure of the fluid by controlling the circulating pump, such that the first layer is cultured with vascular endothelial cells and the second layer is cultured with smooth muscle cells, and
the vascular endothelial cells are arranged such that the flow direction of the fluid becomes the long axis, and the smooth muscle cells are arranged such that a direction perpendicular to the flow direction of the fluid becomes the long axis.

4. The method of claim 1, wherein, in the circulating the fluid,
the solution in the core layer is discharged from the blood vessel tissue along with the fluid such that the blood vessel tissue becomes a tubular blood vessel tissue.

5. The method of claim 1, wherein the method, before the printing a blood vessel mimic, further comprises printing a lower structure of a chamber into which the blood vessel mimic is received; and
in the printing a blood vessel mimic, printing the blood vessel mimic on the lower structure.

6. The method of claim 5, wherein the lower structure comprises a seating part on which the blood vessel mimic is seated, and
in the printing a blood vessel mimic, the blood vessel mimic is printed such that both ends of the blood vessel mimic protrude from the seating part to the outside of the seating part.

7. The method of claim 6, wherein the method further comprises printing, on the lower structure and on the blood vessel mimic, an upper structure of the chamber comprising a fixing part which is extended from the seating part such that both ends of the blood vessel mimic are fixed to the seating part.

8. The method of claim 7, wherein, in the printing an upper structure of the chamber, the fixing part is printed such that both ends of the blood vessel mimic protrude to the outside of the fixing part.

9. The method of claim 8, wherein the lower structure further comprises a lower frame that encompasses both ends of the blood vessel mimic along with the seating part, and the upper structure further comprises an upper frame which is extended from the lower frame and encompasses both ends of the blood vessel mimic along with the fixing part.

10. The method of claim 9, wherein the method further comprises filling a filling material for fixing the blood vessel mimic into a space, which is encompassed with the lower frame, the upper frame, the seating part, and the fixing part.

11. The method of claim 10, wherein the filling material is silicone oil.

12. The method of claim 10, wherein the method, after the filling material is filled, further comprises hardening of the filling material.

13. The method of claim 12, wherein the method further comprises forming, on the cured filling material, a hole to be connected to both ends of the blood vessel mimic, and wherein, in connecting the tubes, the tubes are inserted into the hole and connected to both ends of the blood vessel mimic.

14. (canceled)

15. (canceled)

16. A blood vessel mimic, which comprises:
a first layer, which is printed so as to have a tubular shape using a first bioink in which vascular endothelial cells are mixed with a decellularized extracellular matrix isolated from a blood vessel tissue; and
a second layer, which is printed so as to encompass a side of the first layer and have a tubular shape using a second bioink in which smooth muscle cells are mixed with a decellularized extracellular matrix isolated from a blood vessel tissue,
wherein the first layer and the second layer are crosslinked by calcium ions dissolved in a solution printed together into the space encompassed by the first layer.

17. (canceled)

18. The blood vessel mimic of claim 16, wherein the first bioink and the second bioink further comprise alginate; and
the calcium ions react with the alginate and thereby the first layer and the second layer are crosslinked.

19. The blood vessel mimic of claim 16, wherein, after the first layer and the second layer are crosslinked, the solution in which calcium ions are dissolved is removed by the fluid that flows through the first layer.

20. The blood vessel mimic of claim 16, wherein the solution in which calcium ions are dissolved, the first layer, and the second layer are printed through multiple coaxial nozzles; and
the multiple coaxial nozzles comprise:
a first nozzle, in which the solution where the calcium ions are dissolved is extruded;
a second nozzle, which is arranged concentrically to encompass the first nozzle and in which the first bioink is extruded; and
a third nozzle, which is arranged concentrically to encompass the second nozzle and in which the second bioink is extruded.
